(19) United States
(12) Patent Application Publication
(10) Pub. No.: US 2026/0191452 A1
(43) Pub. Date: Jul. 9, 2026

(54) TEXTILE ELECTRONIC SYSTEM FOR INTEGRATING MUSCLE FATIGUE DETECTION AND TREATMENT

(71) Applicant: Zhejiang University, Hangzhou (CN)

(72) Inventors: Kaichen Xu, Hangzhou (CN); Chongyi Xu, Hangzhou (CN); Huayu Luo, Hangzhou (CN); Ziguan Jin, Hangzhou (CN); Geng Yang, Hangzhou (CN); Huayong Yang, Hangzhou (CN)

(21) Appl. No.: 19/437,250

(22) Filed: Dec. 30, 2025

(30) Foreign Application Priority Data
Jan. 3, 2025 (CN) 202510010822.6

(51) Int. Cl. A61B 5/27; A61B 5/296; A61B 5/395

(52) U.S. Cl. CPC A61B 5/27; A61B 5/296; A61B 5/395

(57) ABSTRACT

A textile electronic system for integrating muscle fatigue detection and treatment includes following components that are integrated on a textile substrate: conductive textile wires, physiological electrodes, vertical interconnect accesses (VIAs) and a surface-mounted device (SMD). The conductive textile wires are arranged on two sides of the textile substrate and communicated with each other through the VIAs, and an integrally formed circuit includes an electrode array and a signal conditioning circuit. The electrode array is configured for detecting an electromyography (EMG) signal and for applying electrical muscle stimulation (EMS), and the signal conditioning circuit is configured for EMG signal amplification, EMS waveform conditioning and overall timing logic control, and has functions of safety isolation and EMS waveform detection. The present disclosure can highly integrate components and devices on a single piece of textile through process design, and realize EMG signal collection and applying EMS.

### CROSS-REFERENCE TO RELATED APPLICATION

This application claims priority of Chinese Patent Application No. 202510010822.6, filed on January 3, 2025, the entire contents of which are incorporated herein by reference.

### TECHNICAL FIELD

The present disclosure belongs to the technical field of intelligent electronic textiles, and particularly relates to a textile electronic system for integrating muscle fatigue detection and treatment.

### BACKGROUND

Textiles with electronic components provide a portable and personalized method for health monitoring and treatment. However, existing solutions often fail to integrate layered circuits and high-density chips on or within textiles, and often require reliance on external commercial printed circuit boards (PCB), which hinders system-level portability and wearing comfort.

The present disclosure uses a laser hybrid manufacturing process to achieve close integration of double-layer circuits, electrodes and flexible sensors on a single piece of textile, which helps to achieve wireless, wearable, breathable health electronic systems. Based on this, the present disclosure further designs a wireless, monolithic integrated, breathable wearable textile electronic system in response to the integrated needs of detection and treatment in precision medicine, which can simultaneously realize the functions of monitoring and relieving muscle fatigue. The overall area of the circuit in the textile electronic system can be less than 50 cm², and it can be worn on human parts such as biceps brachii, quadriceps femoris, etc. to play a role.

Compared with the existing technologies, the present disclosure can realize signal processing and wireless transmission without the need for an external commercial PCB, has good breathability and wearing comfort. The textile electronic system can not only detect physiological signals, but also apply electrical muscle stimulation (EMS) to muscles, relieving muscle fatigue and achieving the integration of detection and treatment.

### SUMMARY

The purpose of the present disclosure is to provide a textile electronic system for integrating muscle fatigue detection and treatment to address the shortcomings of the existing technologies. The textile electronic system integrates functional textiles, flexible electronic components, rigid electronic components, conductive textile wires and physiological electrodes on two sides of a single piece of textile. The textile electronic system can integrate detection and treatment and has great potential in closed-loop diagnosis and treatment.

The present disclosure provides the following technical solutions.

A textile electronic system for integrating muscle fatigue detection and treatment includes following components that are integrated on a textile substrate: conductive textile wires, physiological electrodes, vertical interconnect accesses (VIAs) and surface-mounted device (SMD). The conductive textile wires are arranged on two sides of the textile substrate and communicated with each other through the VIAs, and an integrally formed circuit includes an electrode array and a signal conditioning circuit. The electrode array is configured for detecting an electromyography (EMG) signal and for applying EMS, and the signal conditioning circuit is configured for EMG signal amplification, EMS waveform conditioning and overall timing logic control, and has functions of safety isolation and EMS waveform detection.

Further, in the above solution, the electrode array is constructed by multiplexing a single pair of electrodes, and the electrode array includes a first electrode, a second electrode, a third electrode and a fourth electrode. The first electrode and the second electrode are respectively a positive input terminal and a negative input terminal of the EMG signal, the third electrode is a positive output terminal of EMS, and fourth electrode serves as both a negative output terminal of the EMS and an output terminal of a right leg driving (RLD). Electrodes used for signal sampling are separated from electrodes used for applying EMS to avoid signal sampling being affected by electrode polarization.

Further, the signal conditioning circuit uses three independent optocoupler isolators and an H-bridge circuit composed of four optocoupler isolators, wherein the first electrode, the second electrode, and the fourth electrode are respectively connected to a peripheral circuit through one independent optocoupler isolator, and the third electrode and the fourth electrode are connected to the peripheral circuit through the H-bridge circuit; by controlling all optocoupler isolators, the first electrode, the second electrode, the third electrode and the fourth electrode are selectively connected to the peripheral circuit, so that switching between EMG detection mode and EMS mode is realized.

Further, the signal conditioning circuit uses a constant current source to output stimulation current, amplitude of the stimulation current is controlled by a digital-to-analog converter of a microcontroller, and a direction of the stimulation current is controlled by an H-bridge circuit at a load end of the constant current source; duty cycle, frequency, and amplitude of the stimulation current are all adjustable in real time to ensure an output of electrically neutral current.

Further, in EMS mode, the first electrode, the second electrode, and the fourth electrode are each connected to one independent optocoupler isolator and are all blocked, and the optocoupler isolators in the H-bridge circuit are switched on and off in an orderly manner; in EMG detection mode, the optocoupler isolators in the H-bridge circuit are blocked, and the first electrode, the second electrode, and the fourth electrode are each connected to one independent optocoupler isolator and are all normally open.

Further, the VIAs are portion on the textile substrate where VIAs need to be formed to connect circuits on two sides of textile, and are formed by brushing conductive paste on two sides of a required position by using a pneumatic perfusion method, and respectively blowing air through an air gun at two sides to enable the conductive paste to penetrate into a texture structure of the textile substrate at the required position, so that a front side and a back side of corresponding areas of the textile substrate are communicated.

Further, the conductive textile wires and physiological electrodes are formed by using laser to cut a conductive textile according to a pre-designed pattern, and then the conductive textile wires and the physiological electrodes are transferred and printed to two sides of the textile substrate by a transfer printing method, and the conductive textile wires and the physiological electrodes on the two sides are communicated through the VIAs.

Further, the SMDs is soldered on the conductive textile wire using solder paste to form a complete circuit.

Further, a lower surface of the textile substrate is sequentially arranged with a conductive textile wires and physiological electrode layer, and a medical double-sided adhesive tape and hydrogel layer; the medical double-sided adhesive tape and hydrogel layer is formed by laser cutting the medical double-sided adhesive tapes to form grooves with the same shape as each physiological electrode, injecting a hydrogel precursor into the grooves and curing the grooves with ultraviolet light; the conductive textile wires and the SMDs are distributed on an upper surface of the textile substrate.

The present disclosure has the following beneficial effects.

The disclosed solution realizes the integrated integration of rigid electronic devices such as SMDs, flexible electronic devices such as physiological electrodes, inner and outer double-layer conductive textile wires, and VIAs on the same textile. Moreover, since the independently wired double-layer conductive circuits are realized on a single piece of textile, the solution can allow a circuit layout with a higher degree of integration compared with the existing technologies; and at the same time, it can ensure that the corresponding textile electronic system has the characteristics of breathability, wearability, and treatment safety.

The disclosed solution can simultaneously realize the functions of detecting and relieving muscle fatigue without the need for an external commercial PCB, and is used for integrated detection and treatment of muscle fatigue.

### BRIEF DESCRIPTION OF THE DRAWINGS

FIG. 1 is a geometry diagram and cross-section view of an electrode array of a textile electronic system in the present disclosure.

FIG. 2 is a schematic diagram of a working mode of the electrode array of the textile electronic system in the present disclosure.

FIG. 3 is a three-dimensional (3D) exploded view of the textile electronic system in the present disclosure.

FIG. 4A is a physical diagram of the textile electronic system in the present disclosure.

FIG. 4B is a wearing effect diagram of the textile electronic system in the present disclosure.

FIG. 5 is a program logic diagram of the textile electronic system in the present disclosure in EMG detection mode and EMS mode.

FIG. 6 is a functional block diagram of the textile electronics system in the present disclosure.

FIG. 7A is a current generated by the textile electronic system in the present disclosure when applying EMS to the biceps brachii.

FIG. 7B is a voltage generated by the textile electronic system in the present disclosure when applying EMS to the biceps brachii.

FIG. 7C shows an EMG signal generated during contraction of the biceps brachii.

FIG. 7D shows the power spectral density (PSD) of the EMG signal.

FIG. 8A is a comparison of current consumption between the textile electronic system in the present disclosure and a commercial PCB with the same function in different modes.

FIG. 8B is a waveform of EMS applied by the textile electronic system in the present disclosure.

FIG. 8C shows a waveform of EMS applied by the commercial PCB.

FIG. 9A is an EMG signal collected by the textile electronics system in the present disclosure when a load of 5kg is applied to the biceps brachii.

FIG. 9B shows a root mean square (RMS) of the EMG signal.

FIG. 9C shows a short-time Fourier transform spectrum of the EMG signal.

FIG. 10A shows a median frequency (MDF) of the EMG signals during an application of a load of 5 kg to the biceps brachii and after 10 s of calm rest. The statistical interval is 5 to 160 Hz.

FIG. 10B shows the MDF of the EMG signals during the application of a load of 5 kg to the biceps brachii and after 10s of EMS. The statistical interval is 5 to 160 Hz.

FIG. 11 shows a relationship between maximum voluntary contraction (MVC) force and time during the fatigue recovery of biceps brachii.

### DETAILED DESCRIPTION

The textile electronic system of the present disclosure is further described below with reference to the drawings and embodiments.

The textile electronic system for integrated muscle fatigue detection and treatment is formed on the same textile substrate based on laser hybrid manufacturing process, pneumatic perfusion process, transfer printing combined with welding and other processes. The textile electronic system integrates conductive textile wires, physiological electrodes, VIAs and SMDs, the conductive textile wires are arranged on two sides of the textile substrate and are communicated with each other through the VIAs, that is, a double-layer circuit structure is formed on a single layer of textile. The integrally formed circuit includes an electrode array and a signal conditioning circuit. According to a specific embodiment of the present disclosure, there is also a wireless transmission module in the circuit, and the system also includes an upper computer. The electrode array is configured to detect EMG signals, and an MDF of the EMG signal is used for evaluating the fatigue degree of the muscle. At the same time, the physiological electrodes are configured to apply EMS therapy, so that an MVC force of the muscle can be recovered more quickly. The signal conditioning circuit fully considers the constraints of the textile electronic manufacturing process and the miniaturization needs of wearable devices, is configured for EMG signal amplification, EMS waveform conditioning and overall timing logic control, and has the functions of safety isolation and EMS waveform detection.

The specific circuits in the textile electronic system of the present disclosure can be designed accordingly according to the realized functions. The textile substrate in the textile electronic system is one or more of knitted Modal, medical non-woven, woven polyimide or woven Taffeta, etc. The VIAs are parts where VIAs need to be formed on the textile substrate according to the design drawings to connect the circuits on two sides. Specifically, a pneumatic perfusion method is used to brush conductive paste on both sides of the required portion. Air guns are blown on both sides respectively to allow the conductive paste to penetrate into the texture structure of the textile substrate at this portion, so that the corresponding areas of the textile substrate can be connected on the front and back sides. The conductive paste used is preferably an intermetallic compound liquid metal/copper nanoparticle (LM/Cu NP) paste formed by mixing LM and Cu NPs in a centrifugal mixer or a high-speed defoaming machine. The tangential force generated during the centrifugal process is used to destroy the oxide film on the surface of the LM, so that the LM and Cu NP can fully react during the mixing process. There is no need to introduce additional chemical reagents such as acid and alkali, and the mixture is evenly mixed and the effect is excellent. Laser is used to cut the conductive textile according to a pre-designed pattern to form conductive textile wires and physiological electrodes, and then the conductive textile wires and the physiological electrodes are transferred and printed to two sides of the textile substrate by a transfer printing method, and the conductive textile wires and the physiological electrodes on both sides are communicated through the VIAs. Specifically, the following methods can be used: pressing an adhesive film (AF) on one side of a metal textile (MT), and pressing a sticky water-soluble sacrificial layer (SL) on the other side of the MT to form an AF/MT/SL structure. Using an ultraviolet nanosecond laser to cut the AF and MT into a patterned conductive circuit, while the SL remains intact, attaching the entire AF/MT/SL to the textile substrate with the AF side, then immersing it in water, and removing the SL layer after softening in water. The AF/MT layer attached to the textile substrate is dried, and the excess AF/MT layer except the patterned conductive circuit is peeled off to form conductive textile wires and physiological electrodes. Solder paste is used to solder the SMDs onto conductive textile wires to form a complete circuit. The solder paste is preferably a low-melting point solder paste formed by mixing tin-based paste and aluminum flux, which effectively removes the nickel oxide layer of commercial SMDs to ensure the welding effect.

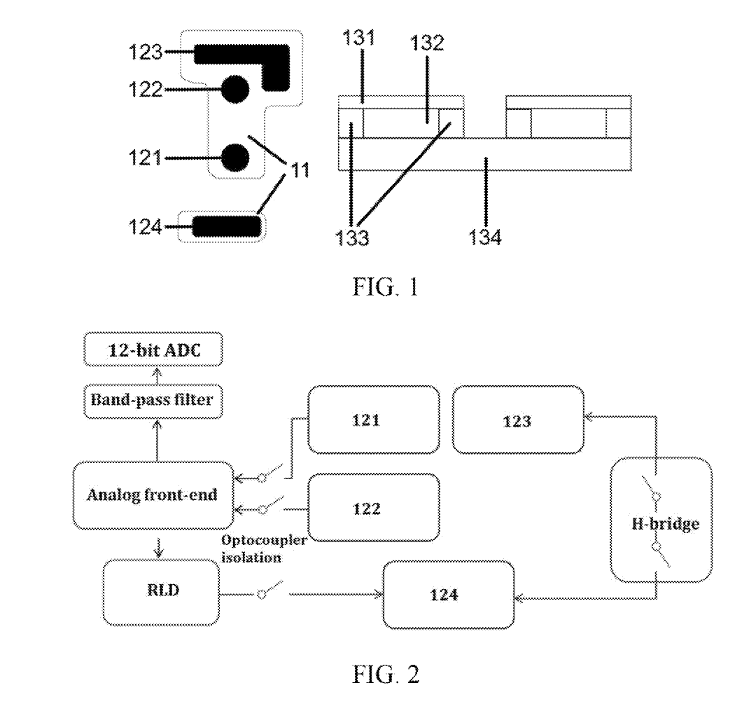

As shown in FIGS. 1-11, a specific embodiment of the present disclosure is disclosed. In this embodiment.

(1) The electrode array is constructed by using a single-pair of electrode multiplexing solution: electrodes 121, 122, and 124 are used to detect EMG signals, and electrodes 123 and 124 are used to apply EMS. The multiplexing of electrode 4 is realized through an optocoupler isolator, and electrode multiplexing effectively saves area.

(2) Design of electrical stimulation source for textile-based signal conditioning circuits: a constant current source is used to output stimulation current. The constant current source is powered by a single-stage boost circuit in the BOOST topology, which converts the 3.7 V lithium battery input voltage to a 40 V output voltage. The amplitude of the stimulation current is controlled by the DAC (digital-to-analog converter) of the STM32L052 microcontroller. The direction of the stimulation current is controlled by the H-bridge at the load end of the constant current source, which consists of 4 optocoupler isolators. Therefore, the duty cycle, frequency, and amplitude of the stimulation current can be adjusted in real time to ensure the output of electrically neutral current, thereby ensuring the comfort and safety of treatment.

(3) Signal acquisition design of the textile-based signal conditioning circuit: The ADC (analog-to-digital converter) of STM32L052 is used to collect the EMG signals and EMS waveforms, and the direct memory access (DMA) function of STM32L052 is used to control the sampling and subsequent data transmission of the ADC to improve the real-time performance of the program. At the same time, the sampling rate is switched according to the different sampling signals: when collecting the EMS waveform, since the EMS waveform is a sparse short pulse (cycle 20ms, and pulse duration 3ms), the ADC in the EMS mode only performs high-rate sampling (40 kHz) within 3ms after the pulse occurs, and does not sample the rest of the time, thereby reducing the occupation of storage and transmission bandwidth; when collecting the EMG signal, the ADC continuously performs low-rate sampling (720 Hz) and synchronously transmits the signals. In order to improve the signal-to-noise ratio of the EMG signal before sampling, an instrumentation amplifier and a band-pass filter are used to amplify the signal at the electrode terminal, a RLD is used to suppress common-mode noise from the human body, and a πRC network is used to filter out power ripple noise caused by electrical stimulation modules and Bluetooth modules.

(4) Safe isolation design of textile-based signal conditioning circuit: During applying EMS, in order to avoid the connection between the low-voltage EMG signal sampling electrodes and the high-voltage electrical stimulation electrodes on the skin surface, an optocoupler isolator is designed at the connection between the circuit and each electrode. In the EMS mode, the optocoupler of the EMG signal sampling electrode is blocked, and the optocoupler of the EMS electrode is switched on and off in an orderly manner; in the EMG mode, the optocoupler of the EMS electrode is blocked, and the optocoupler of the EMG signal sampling electrode is normally open.

### Specific embodiments are described as follows.

FIG. 1 shows a schematic structural diagram of the electrode array in this example. As shown on the left side of FIG. 1, the first electrode 121 and the second electrode 122 are positive and negative input terminals of EMG signals respectively. The third electrode 123 is a positive output terminal of EMS, and the Fourth electrode 124 serves as both a negative output terminal of EMS and an output terminal of the RLD. The right side of FIG. 1 is a schematic diagram of the contact interface between the electrode and the skin. The electrode 131 is bonded to the human skin 134 through a hydrogel 132 and a medical double-sided adhesive tape 133. Among them, the EMS current output by electrodes 123 and 124 is too high, which can easily cause polarization on the electrode surface, not only reducing the working efficiency of the electrode, but also causing a significant increase in impedance. In order to ensure the accuracy and stability of signal acquisition, the EMG signal sampling electrodes (electrodes 121 and 122) require low impedance, so the EMG signal sampling electrodes (electrodes 121 and 122) and the output EMS electrodes (electrodes 123 and 124) need to be strictly separated. The RLD has lower impedance requirements and is less affected by polarization, so the electrode 124 can be used as both the negative output terminal of EMS and the output terminal of the RLD. The four electrodes are compactly arranged on the textile 11, and the multiplexing of the Fourth electrode 124 effectively improves space utilization. The shape of the above electrodes can be adjusted according to the needs of the scene. As shown in the cross-section on the right side of FIG. 1, conductive hydrogel is used at the contact interface between the electrode and the skin to reduce the skin-electrode impedance. The medical double-sided adhesive tape is placed on the periphery of the hydrogel to ensure that the electrode can be stably attached to the skin surface during wearing. Although using Ag/AgCl with low polarization impedance as the electrode material can avoid the influence of electrode polarization and realize the multiplexing of the EMG signal sampling electrodes (electrodes 121 and 122) and the output EMS electrodes (electrodes 123 and 124), in order to improve the versatility of materials and easily process patterned electrodes, thereby facilitating circuit layout, the present disclosure selects metallized textiles and hydrogels as the electrode materials.

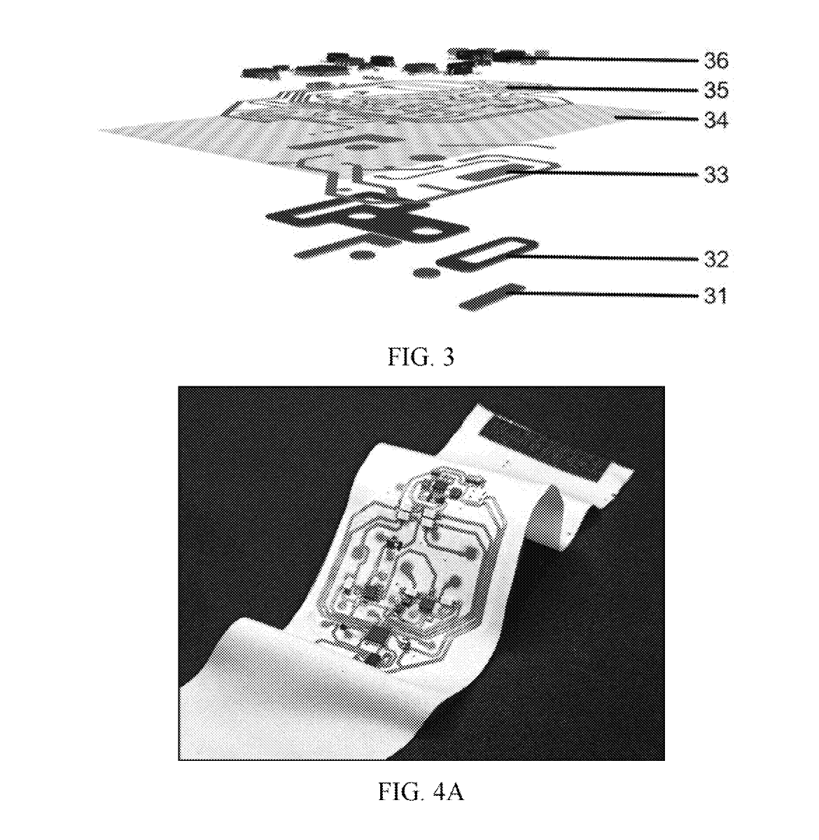

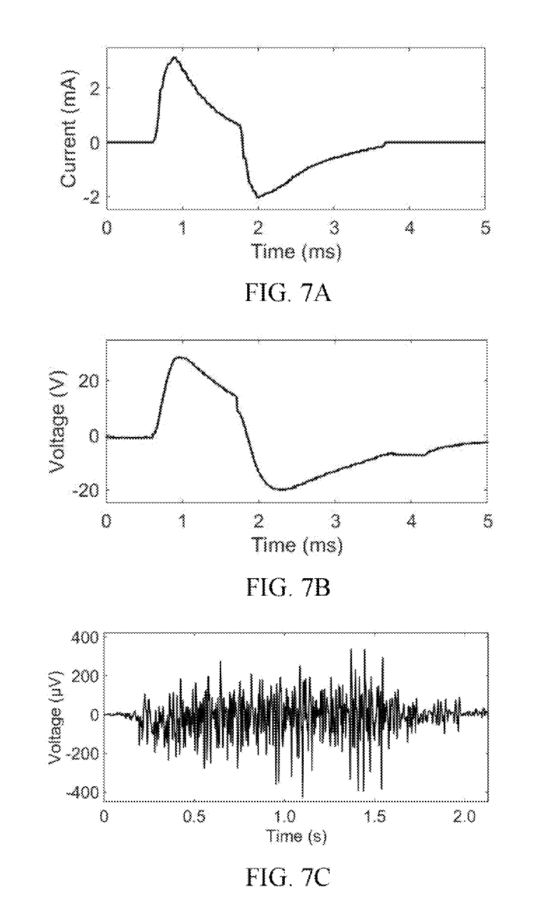

FIG. 2 shows a schematic diagram of the working mode of the electrode array, and FIG. 6 is a complete functional block diagram of the textile electronic system in this specific example. By configuring the optocoupler isolator and the H-bridge circuit, the electrodes are selectively connected to the peripheral circuit, thereby achieving different working modes. Among them, the H bridge consists of 4 optocoupler isolators. In the EMG detection mode, the optocoupler inside the H bridge on the right side of the FIG. 2 is blocked, the three optocouplers on the left side of the FIG. 2 are normally open, and the first, second and fourth electrodes are connected to the peripheral circuits. In EMS mode, the three optocouplers on the left side of the figure are blocked, the optocoupler inside the H-bridge on the right side of the figure is switched on and off in an orderly manner, and the third and fourth electrodes are connected to peripheral circuits. Since the peripheral circuits on the left side of the figure have low voltages of 3.3V and below, while the peripheral circuits on the right side of the figure have high voltages of 40V, adding optocoupler isolation to the electrodes can prevent high and low voltages from being shorted on the skin surface and play a protective role.

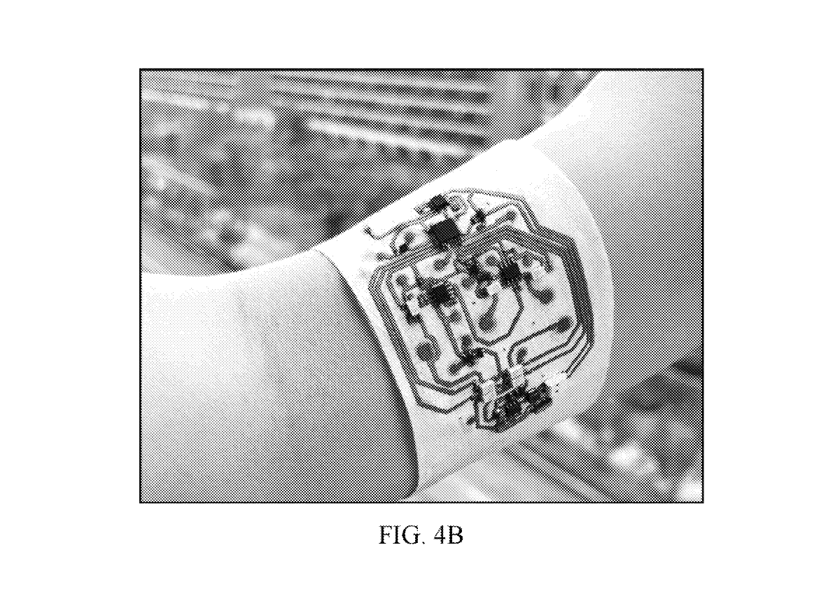

FIG. 3 shows a 3D exploded view of the entire textile electronic system. From bottom to top, there are hydrogel 31, medical double-sided adhesive tape 32, a bottom textile circuit and electrode array 33, a textile substrate 34, a top textile circuit 35, and a SMD 36. Among them, the medical double-sided adhesive tape 32 is cut into a specific shape by laser, and the hydrogel 31 is obtained by dripping precursor liquid into a groove surrounded by the medical double-sided adhesive tape 32 and curing with ultraviolet light.

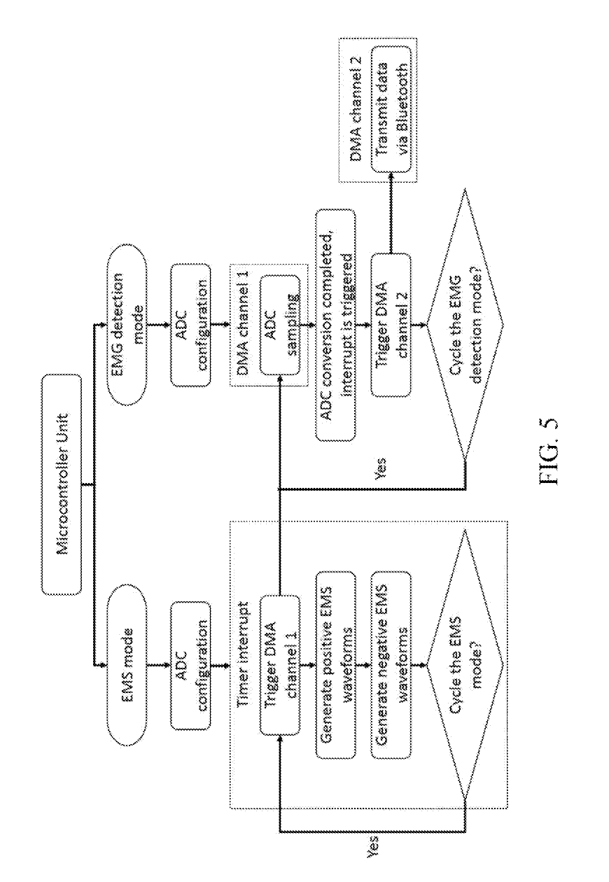

As shown in FIGS. 4A and 4B, FIG. 4A is a physical diagram of the textile electronic system, and FIG. 4B is the wearing effect diagram of the textile electronic system on the biceps brachii of the arm. It can be seen that the electronic system has good bendability and high space utilization.

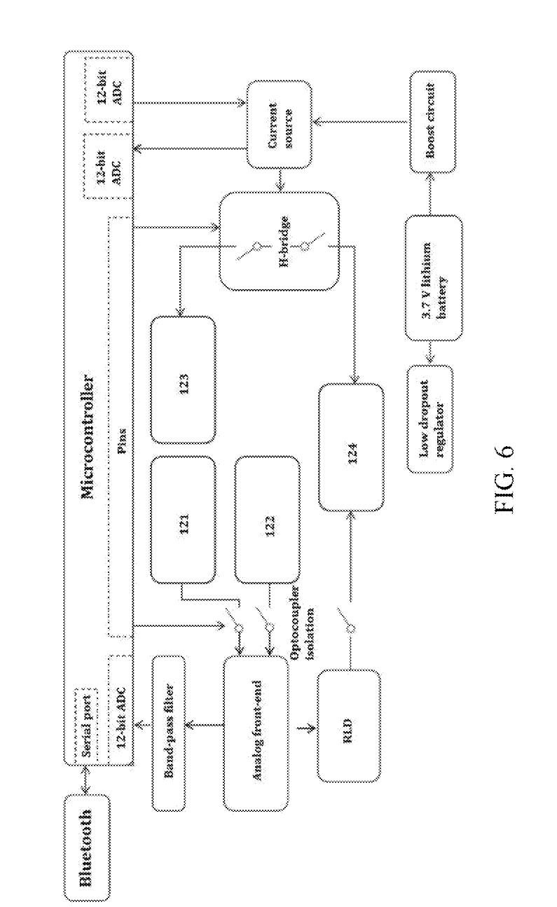

FIG. 5 shows the program logic diagram of the textile electronic system that can be used in EMG detection mode and EMS mode. EMS is controlled by a timer interrupt with a cycle of 20 ms, and it is determined whether to proceed with the next cycle at the end of the interrupt. This process first triggers DMA channel 1, allowing the ADC to sample the EMS to be applied (pulse duration 3 ms), and then immediately generates positive and negative EMS without sampling during the remaining 17 ms of the cycle, thereby reducing the occupation of storage space and transmission bandwidth. After DMA channel 1 completes a round of sampling, a DMA channel 2 is triggered immediately, and the DMA channel 2 transmits the collected EMS waveform to the Bluetooth module, and finally the Bluetooth module transmits the signals to the upper computer. EMG detection is completed through cyclic execution of DMA channel 1 and DMA channel 2. This process first triggers DMA channel 1, allowing the ADC to sample the EMG signal. After the ADC conversion is completed, DMA channel 2 is immediately triggered. At the same time, the next round of EMG signal sampling is carried out. DMA channel 2 transmits the collected EMG signal to the Bluetooth module, which finally transmits the waveform to the upper computer. Among them, the use of DMA in the two modes allows the textile electronic system to simultaneously collect and transmit signals, greatly improving data transmission efficiency and real-time performance.

FIG. 6 shows a functional block diagram of the textile electronic system. In the EMG detection mode, the EMG signals detected by the three electrodes (electrodes 121, 122, and 124) are processed through analog front-end and band-pass filtering, and then collected by the 12-bit ADC channel of the microcontroller, and finally transmitted through the Bluetooth module to the upper computer. In the EMS mode, the EMSs current is output through a current source, in which the current source is boosted by a 3.7V lithium battery to 40V and then supplies power. The amplitude of the EMS current is controlled by the DAC of the microcontroller, and the direction of the EMS current is controlled by the H-bridge. The EMS currents detected by the three electrodes (electrodes 121, 122, and 124) are processed through analog front-end and band-pass filtering, and then collected at a high rate by the 12-bit ADC channel of the microcontroller during the electrical stimulation pulse time, and finally transmitted to the upper computer via the Bluetooth module.

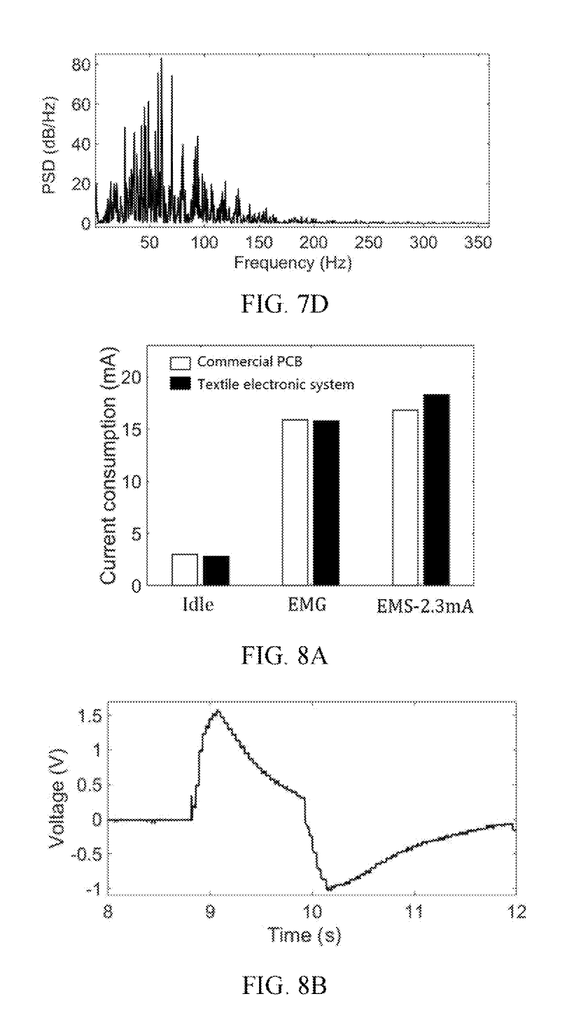

As shown in FIGS. 7A, 7B, 7C, and 7D, the applied EMS and the detected EMG signals of the textile electronic system are analyzed. FIG. 7A shows the EMS waveform generated by the textile electronic system. The EMS current pulse has bipolar and electrically neutral characteristics, which avoids the accumulation of charges on the skin and enhances the comfort and safety of applying EMS. FIG. 7B shows the voltage of the EMS. FIG. 7C shows the EMG signal collected during the contraction of the biceps brachii. The signal-to-noise ratio is high and the distribution of PSD of the EMG signal is normal (FIG. 7D).

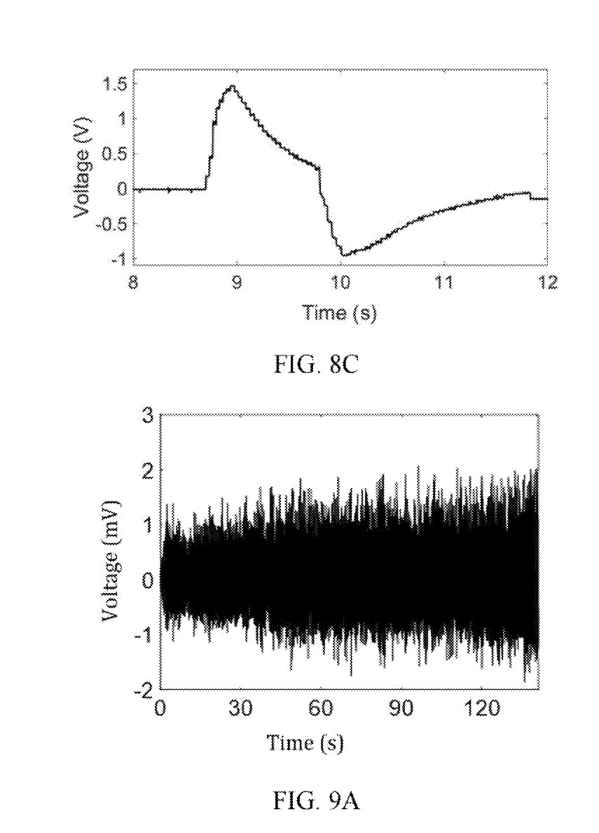

As shown in FIG. 8A and FIG. 8B, the textile electronic system is compared to a commercial PCB. FIG. 8A shows the current consumption of the textile electronic system and the commercial PCB in idle mode (idle), EMG detection mode, and EMS mode respectively. FIGS. 8B and 8C show the voltage waveforms applied by the textile electronic system and the commercial PCB board on a 1 kΩ resistor respectively. Similar current consumption and almost identical EMS waveforms demonstrate the reliability of the textile electronic system.

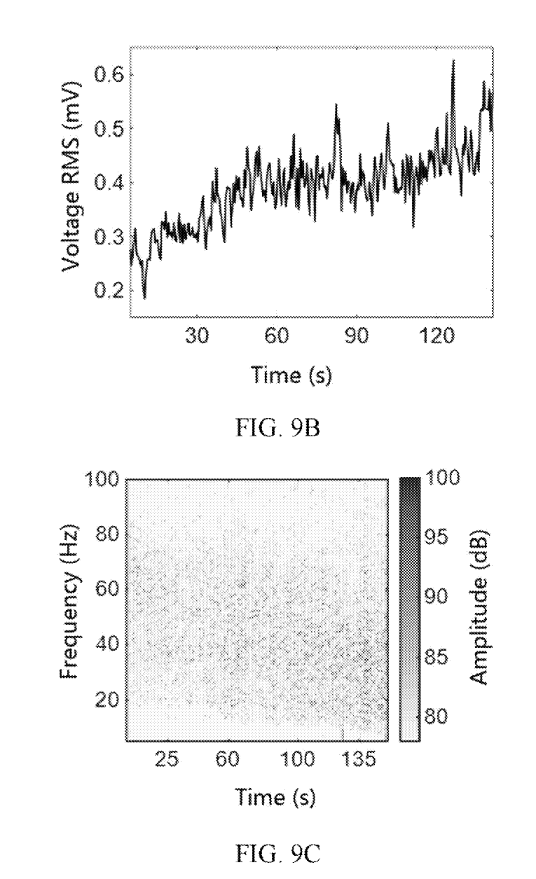

As shown in FIGS. 9A, 9B, and 9C in order to verify the ability of the textile electronic system to detect muscle fatigue, the textile electronic system is worn on the biceps brachii of a volunteer and loaded with a weight of 5 kilograms for 2.5 minutes. FIG. 9A shows the EMG signal collected during the above-mentioned loading process, and FIGS. 9B and 9C show the RMS amplitude and short-time Fourier transform spectrum of the EMG signal respectively. The increase in the root-mean-square amplitude of EMG signal over time is caused by increased neural recruitment caused by gradual muscle fatigue. The shift of the frequency distribution of the short-time Fourier transform spectrum of EMG signals to low frequencies over time is caused by the decrease in action potential conduction speed caused by gradual muscle fatigue. This proves the ability of the textile electronic system to detect muscle fatigue.

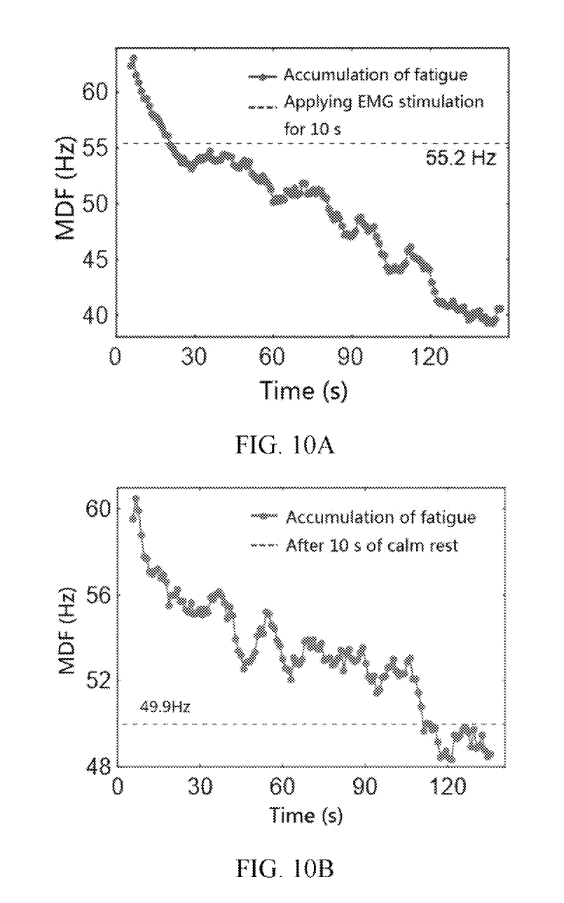

As shown in FIGS. 10A and 10B, to illustrate the ability of the textile electronic system to treat muscle fatigue, the textile electronic system is worn on the biceps brachii of a volunteer and loaded with a weight of 5 kilograms for 2.5 minutes to accumulate fatigue in the muscles. Then the EMS mode of the textile electronic system is turned on for 10 seconds. The MDF of the EMG signal during the entire experiment is as shown in FIG. 10A. For comparison, two days later, the volunteer performed the weight-bearing experiment again and rests calmly for 10 seconds. The MDF of the EMG signal during the entire experiment is shown in FIG. 10B. The lower the MDF value, the higher the degree of muscle fatigue. During 10 seconds of calm rest, MDF value is recovered only 11%, whereas after applying EMS for 10 seconds, the MDF value is recovered nearly 65%. It is demonstrated that the textile electronic system has the potential to accelerate the recovery of muscle bioelectrical activity.

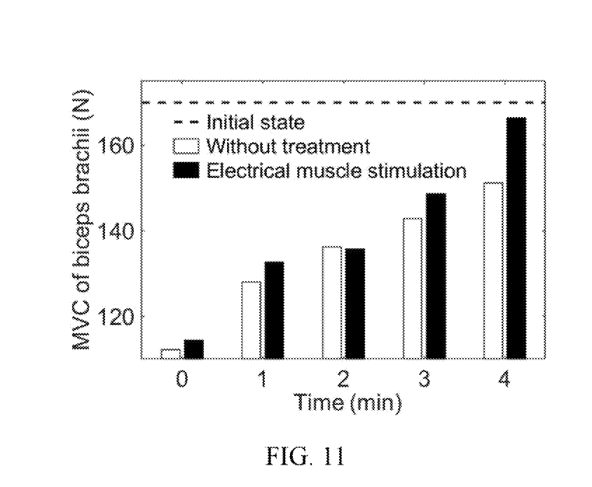

In order to further verify the ability of the textile electronic system to treat muscle fatigue, the MVC force of muscles is used to quantify the degree of muscle fatigue: the lower the MVC value, the higher the degree of muscle fatigue. The MVC measurement method is as follows: the biceps brachii pulls the force sensor through the rope and fixed pulley for 3 seconds, and the maximum reading of the force sensor is regarded as the MVC. Subsequently, a volunteer (with an initial MVC of 170 N for the biceps brachii) is asked to continuously pull the force sensor through the above apparatus. When the sensor reading remains below 100 N for a continuous duration of 5 seconds, it is determined that the biceps brachii is exhausted. After exhaustion, calm rest or applying EMS is performed to relieve muscle fatigue, and the MVC value is measured every minute. As shown in FIG. 11, after applying EMS, MVC is returned to 98% of its original state within 4 minutes. In contrast, in the control group with calm rest, MVC is only returned to 89% of its original state. This demonstrates that the textile electronic system of the present disclosure can accelerate the recovery of muscle strength.

1. A textile electronic system for integrating muscle fatigue detection and treatment, comprising following components that are integrated on a textile substrate: conductive textile wires, physiological electrodes, vertical interconnect accesses (VIAs) and a surface-mounted device (SMD), the conductive textile wires being arranged on two sides of the textile substrate and communicated with each other through the VIAs, and an integrally formed circuit comprising an electrode array and a signal conditioning circuit, wherein the electrode array is configured for detecting an electromyography (EMG) signal and for applying electrical muscle stimulation (EMS), and the signal conditioning circuit is configured for EMG signal amplification, EMS waveform conditioning and overall timing logic control, and has functions of safety isolation and EMS waveform detection;
the VIAs are formed by brushing conductive paste on two sides of a required portion by using a pneumatic perfusion method, and blowing air through an air gun at two sides to enable the conductive paste to penetrate into a texture structure of the textile substrate at the required portion, causing a front side and a back side of corresponding areas of the textile substrate to be communicated, wherein the conductive paste is an intermetallic compound liquid metal/copper nanoparticle (LM/Cu NP) paste formed by mixing LM and Cu NPs in a centrifugal mixer or a high-speed defoaming machine.

2. The textile electronic system of claim 1, wherein the electrode array is constructed by multiplexing a single pair of electrodes, and the electrode array comprises a first electrode, a second electrode, a third electrode and a fourth electrode, wherein the first electrode and the second electrode are respectively a positive input terminal and a negative input terminal of the EMG signal, the third electrode is a positive output terminal of EMS, and fourth electrode serves as both a negative output terminal of the EMS and an output terminal of a right leg driving (RLD); and electrodes used for signal sampling are separated from electrodes used for applying EMS to avoid signal sampling being affected by electrode polarization.

3. The textile electronic system of claim 2, wherein the signal conditioning circuit uses three independent optocoupler isolators and an H-bridge circuit composed of four optocoupler isolators, wherein the first electrode, the second electrode, and the fourth electrode are connected to a peripheral circuit through one independent optocoupler isolator, and the third electrode and the fourth electrode are connected to the peripheral circuit through the H-bridge circuit; and by controlling all optocoupler isolators, the first electrode, the second electrode, the third electrode and the fourth electrode are selectively connected to the peripheral circuit, effectively realizing independent switching between EMG detection mode and EMS mode, avoiding signal interference and ensuring system safety.

4. The textile electronic system of claim 3, wherein the signal conditioning circuit uses a constant current source to output stimulation current, amplitude of the stimulation current is controlled by a digital-to-analog converter of a microcontroller, and a direction of the stimulation current is controlled by an H-bridge circuit at a load end of the constant current source; and duty cycle, frequency, and amplitude of the stimulation current are adjustable in real time to ensure an output of electrically neutral current and avoid damage to skin and tissue caused by excessive current.

5. The textile electronic system of claim 3, wherein in the EMS mode, the first electrode, the second electrode, and the fourth electrode are each connected to one independent optocoupler isolator and are all blocked, and the optocoupler isolators in the H-bridge circuit are switched on and off in an orderly manner; and in the EMG detection mode, the optocoupler isolators in the H-bridge circuit are blocked, and the first electrode, the second electrode, and the fourth electrode are each connected to one independent optocoupler isolator and are all normally open.

6. The textile electronic system of claim 1, wherein the conductive textile wires and physiological electrodes are formed by using laser to cut a conductive textile according to a pre-designed pattern, then the conductive textile wires and the physiological electrodes are transferred and printed to two sides of the textile substrate by a transfer printing method, and the conductive textile wires and the physiological electrodes on the two sides are communicated through the VIAs.

7. The textile electronic system of claim 1, wherein the SMD is soldered on the conductive textile wires using solder paste to form a complete circuit.

8. The textile electronic system of claim 1, wherein a lower surface of the textile substrate is sequentially arranged with a conductive textile wire and physiological electrode layer, and a medical double-sided adhesive tape and hydrogel layer; the medical double-sided adhesive tape and hydrogel layer is formed by laser cutting the medical double-sided adhesive tapes to form grooves with the same shape as each physiological electrode, injecting a hydrogel precursor into the grooves and curing the grooves with ultraviolet light; and the conductive textile wires and the SMD are distributed on an upper surface of the textile substrate.
